(19) United States
(12) Patent Application Publication
(10) Pub. No.: US 2007/0196492 A1
(43) Pub. Date: Aug. 23, 2007

(54) Stimuli-Responesive Hydrogel,Production Method Of Stimuli-Responsive Hydrogel,And Polymer Actuator Using Stimuli-Responsive Hydrogel

(75) Inventors: Hidetoshi Ito, Kanagawa (JP); Noritoshi Araki, Kanagawa (JP)

Correspondence Address: BELL, BOYD & LLOYD, LLP, P. O. BOX 1135, CHICAGO, IL, 60690, US

(73) Assignee: SONY CORPORATION, Tokyo (JP)

(21) Appl. No.: 10/599,071

(22) PCT Filed: Mar. 16, 2005
(86) PCT No.: PCT/JP05/05312

§ 371 Date: Sep. 19, 2006

(30) Foreign Application Priority Data
Mar. 19, 2004 (JP) P2004-080537

(51) Int. Cl. A61K 9/14

(52) U.S. Cl. 424/486

(57) ABSTRACT

A stimuli-responsive polymer hydrogel is provided. The stimuli-responsive polymer hydrogel has a high breaking strength and excellent stimuli-responsive function and is highly stable with elapse of time. The stimuli-responsive polymer hydrogel is capable of gelating as a result of absorbing and swelling with water and capable of changing its degree of swelling and/or volume in response to a stimulus and contains a water-insoluble polymer as a phase separation structure.

### CROSS REFERENCE TO RELATED APPLICATIONS

The present application claims priority to Japanese Patent Document No. 2004-080537 filed on Mar. 19, 2005, the disclosures of which are herein incorporated by reference.

### BACKGROUND

The present disclosure relates to stimuli-responsive hydrogels, production methods of the stimuli-responsive hydrogels, and polymer actuators using the stimuli-responsive hydrogels.

Robots have been increasingly received attention in nursing care supports, dangerous works, entertainments, and other various applications.

Robots applied to these applications have multiple joints (moving elements) as in animals, and now they have to realize more complicated operations.

Magnetic motors have been conventionally used as actuators for driving the moving elements of robots. However, these actuators have large weights, because they are made of metals. If actuators having such large weights are mounted to the moving elements of robots, the resulting robots require high power to operate the moving elements, because the weights of actuators apply loads to the moving elements upon operation. On the other hand, such actuators requiring high power must inevitably have large sizes and large weights. Thus, there arises an irresolvable contradiction.

In addition, the use of magnetic motors requires decelerators for controlling number of revolutions and torque at necessary levels, and they are practically disadvantageous in that gears used in the decelerators gradually deteriorate in their performance due to abrasion.

In contrast, ultrasonic motors achieve high torque even when rotated at low speed and thereby do not require decelerators. However, these ultrasonic motors are also made of metallic materials, thereby have large weights and suffer from the above-mentioned problems.

Accordingly, attention has therefore been focused on “polymer actuators” that comprise polymer materials, have low weights, are flexible, and can avoid decreased performance due to abrasion.

Examples of these polymer actuators are polymer piezoelectric elements typically using poly(vinylidene fluoride)s; conductive polymer actuators typically using electronic-conductive polymers; and gel actuators typically using polymer gels.

Of the gel actuators, polymer hydrogel actuators using polymer gels capable of swelling with water utilize the phenomenon that stimuli-responsive polymer hydrogels change their volume in response to the environment such as ambient temperature, ion concentration, and pH. They have a percentage of change as large as 30% to 50% and can exhibit performance equivalent to living skeletal muscles.

However, the temperature is difficult to control, because high-speed control of heating and cooling is difficult. The control of ion concentration requires extra operations such as forced exchange of the ambient solution typically using a pump, and this results in the need of extra tanks for storing electrolytes to be applied. This technique is therefore unsuitable for actuators for use in lightweight, downsized systems.

In contrast, the pH can be changed by the replacement of the ambient solution or by using an electrochemical reaction.

Specifically, the pH can be changed in the following manner. An aqueous electrolyte solution is used as the ambient solution of the stimuli-responsive polymer hydrogel; a voltage is applied between electrodes arranged in the aqueous solution so as to consume hydrogen ion and/or hydroxide ion as a result of electrode reaction or to yield a concentration gradient due to electric double layer occurring on the surfaces of the electrodes, to thereby change the pH in the vicinity of the electrodes. The pH-responsive hydrogel can be electrically controlled and driven by using this phenomenon. The use of the electrical control and electrical drive enables high-speed control of volume change of the stimuli-responsive polymer hydrogel only by means of a power source and a control circuit.

When a stimuli-responsive polymer hydrogel is used as an actuator using its expansion/contraction, the hydrogel must have such a strength as to sufficiently endure drag force acting upon the actuator. If the hydrogel has an insufficient strength, it may undergo contraction break or tensile break due to force generated by the hydrogel itself or the drag force and may fail to work.

However, a hydrogel is generally a material having a low breaking strength against contraction and tension.

This is probably because a polymer constituting the hydrogel has been crosslinked, its molecular chain is thereby limited in motility, and stress cannot be dispersed in the hydrogel.

Techniques for improving the breaking strength of hydrogels include a technique of impregnating a hydrogel with water-soluble monomer, and carrying out crosslinking and polymerization to thereby form two independent crosslinked polymers in the gel (see, for example, Yoshihito Nagata, Proceedings of the Society of Polymer Science, Japan, 51 (2002)3280); and a technique of mixing a hydrogel with a poly(vinyl alcohol) (PVA), subjecting the mixture to heating treatment or freezing/thawing treatment to thereby carrying out gelation by the action of microcrystalline cross-linking points of PVA (see, for example, Makoto Suzuki, KOBUNSHI RONBUNSHU (Japanese Journal of Polymer Science and Technology) 46 (1989) 603).

However, any of these disclosed techniques uses a water-soluble polymer as a “reinforcer”, and a large quantity of the reinforcer must be introduced into the hydrogel in order to sufficiently reinforce the hydrogel in a state of hydration.

The introduction of the reinforcer in a large quantity results in a decreased content of the stimuli-responsive polymer, and this inevitably invites decreased functions with respect to responsiveness to stimuli.

A polymer used as the reinforcer is preferably uncrosslinked, because the breaking strength decreases when the polymer constituting the hydrogel has been crosslinked, as is described above. However, such an uncrosslinked water-soluble polymer, if introduced into a hydrogel, dissolves out of the gel with time and is practically unsuitable.

Accordingly, there is a need to provide a stimuli-responsive polymer hydrogel that has a high breaking strength and excellent stimuli-responsive function and is highly stable with elapse of time; a method for producing the same; and a polymer actuator using the same.

### SUMMARY

The present disclosure provides a stimuli-responsive polymer hydrogel capable of gelating as a result of absorbing and swelling with water and capable of changing its degree of swelling and/or volume in response to a stimulus, wherein the polymer hydrogel comprises a water-insoluble polymer as a phase separation structure.

The present disclosure further provides a method for producing a stimuli-responsive polymer hydrogel, comprising carrying out the polymerization of a monomer having a stimuli-responsive functional group with a crosslinker in a solution of a water-insoluble polymer in an organic solvent to yield an organogel comprising the water-insoluble polymer and a stimuli-responsive polymer; subjecting the organogel to one treatment selected from drying under reduced pressure, drying by heating, and drying by heating under reduced pressure to remove the organic solvent to thereby yield a dried gel; and allowing the dried gel to swell with water to thereby yield a hydrogel.

The present disclosure further provides a method for producing a stimuli-responsive hydrogel, comprising carrying out the polymerization of a monomer having a stimuli-responsive functional group with a crosslinker in a solution of a water-insoluble polymer in an organic solvent to yield an organogel comprising the water-insoluble polymer and a stimuli-responsive polymer; and immersing the organogel in water or a water-containing liquid mixture to thereby yield a hydrogel.

In addition, the present disclosure provides a polymer actuator comprising a stimuli-responsive polymer hydrogel capable of gelating as a result of absorbing and swelling with water and capable of changing its degree of swelling and/or volume in response to a stimulus, the stimuli-responsive polymer hydrogel comprising a water-insoluble polymer as a phase separation structure.

The stimuli-responsive hydrogel according to the present invention has a very excellent breaking strength as a result of incorporating a water-insoluble polymer into the stimuli-responsive polymer hydrogel.

By using a water-insoluble polymer as a “reinforcer”, the reinforcer does not undergo hydration in the hydrogel, can exhibit high reinforcing activity even in a small amount, and can avoid deterioration in stimuli-responsive functions.

The application of the water-insoluble polymer as the reinforcer also prevents the reinforcer from dissolving out even when the hydrogel is placed in water. This eliminates the need of crosslinking the water-insoluble polymer as the reinforcer in order to suppress dissolution out and enables the use of a water-insoluble polymer having no cross-linking point as the reinforcer. Thus, a high breaking strength can be structurally reliably obtained.

The water-insoluble polymer can be brought into a rubbery state upon use by setting the glass transition temperature Tg of the water-insoluble polymer used as the reinforcer being lower than the working temperature of the stimuli-responsive polymer hydrogel. The molecular chain in a rubbery state has higher motility than that in a glassy state, the stress can be more easily dispersed, and the reinforcer can exhibit high reinforcing activity.

As has been discussed above, the stimuli-responsive polymer hydrogel containing a water-insoluble polymer achieves a high breaking strength. Thus, when the hydrogel is used as an actuator, the actuator can excellently work without breakage of the gel. Such breakage of the gel is caused by the volume change of the gel in response to a stimulus, the force formed with the volume change, or the drag force.

Embodiments of the stimuli-responsive hydrogels with production methods thereof, and actuators using the stimuli-responsive hydrogels are described below. It should be noted, however, the followings are only examples and by no means limit the scope of the present invention.

The stimuli-responsive hydrogels according to an embodiment are capable of gelating as a result of absorbing and swelling with water and capable of changing their degree of swelling and/or volume in response to a stimulus, and each comprise a water-insoluble polymer.

Known conventional hydrogel materials that change in degree of swelling in response to a change in the environment such as ambient temperature, ion concentration, or pH can be used as the stimuli-responsive polymer hydrogels.

The temperature can be controlled, for example, by arranging a heater and/or a cooler in the ambient of the stimuli-responsive polymer hydrogel and appropriately adjusting the device. The ion concentration can be controlled, for example, by arranging the stimuli-responsive hydrogel in a predetermined chamber, placing an electrolytic solution in the chamber, and exchanging the electrolytic solution typically with a pump.

The pH can be changed by exchanging an electrolytic solution in the ambient of the stimuli-responsive polymer hydrogel in the above-mentioned manner. Alternatively, it can also be changed using an electrochemical reaction. In this case, the pH can be controlled at high speed only using a power source and a control circuit. Accordingly, the stimuli-responsive hydrogels according to an embodiment are preferably pH-responsive polymer hydrogels in consideration of the convenience for applying the stimuli-responsive polymer hydrogels to actuators.

Examples of the pH-responsive stimuli-responsive polymers are polymers each intramolecularly having an acidic functional group such as a carboxylic acid or a sulfonic acid group, or a basic functional group such as a primary amine, a secondary amine, or a tertiary amine.

Specific examples are polymers as polymerized products of monomers such as acrylic acid, methacrylic acid, vinyl acetate, maleic acid, vinylsulfonic acid, styrenesulfonic acid, vinylpyridine, vinylaniline, vinylimidazole, aminoethyl acrylate, methylaminoethyl acrylate, dimethylaminoethyl acrylate, ethylaminoethyl acrylate, ethylmethylaminoethyl acrylate, diethylaminoethyl acrylate, aminoethyl methacrylate, methylaminoethyl methacrylate, dimethylaminoethyl methacrylate, ethylaminoethyl methacrylate, ethylmethylaminoethyl methacrylate, diethylaminoethyl methacrylate, aminopropyl acrylate, methylaminopropyl acrylate, dimethylaminopropyl acrylate, ethylaminopropyl acrylate, ethylmethylaminopropyl acrylate, diethylaminespropyl acrylate, aminopropyl methacrylate, methylaminopropyl methacrylate, dimethylaminopropyl methacrylate, ethylaminopropyl methacrylate, ethylmethylaminopropyl methacrylate, diethylaminopropyl methacrylate, dimethylaminoethylacrylamide, and dimethylaminopropylacrylamide.

Where necessary, intramolecularly or intermolecularly crosslinked polymers derived from these polymers; copolymers between these monomers and other monomers; and mixtures of these polymers with other polymers can be used.

Known or conventional polymer materials can be used as the water-insoluble polymer to be incorporated into the stimuli-responsive polymer hydrogel. Examples thereof are poly(methyl methacrylate)s, polystyrenes, and poly(vinylidene fluoride)s having appropriately set molecular weights. Each of these can be used alone or in combination.

The water-insoluble polymer is preferably a polymer having no cross-linking point, because the resulting water-insoluble polymer satisfactorily contributes to dispersion of the stress and highly effectively acts to reinforce.

In addition, the water-insoluble polymer used herein preferably has a glass transition temperature Tg lower than the working temperature of the stimuli-responsive polymer hydrogel. This is because, when the water-insoluble polymer stands a rubbery state at the working temperature of the stimuli-responsive polymer hydrogel, the resulting water-insoluble polymer satisfactorily contributes to dispersion of the stress and highly effectively acts to reinforce.

Specifically, when the stimuli-responsive polymer hydrogel is used at around room temperature, the water-insoluble polymer preferably has a glass transition temperature Tg lower than 20° C.

The content of the water-insoluble polymer is not specifically limited. With an increasing content of the water-insoluble polymer, the reinforcing effect increases but, concurrently, the change in degree of swelling of the hydrogel and the change in volume of the gel in response to a stimulus decrease.

Specifically, the breaking strength, degree of swelling, and volume change of the stimuli-responsive polymer hydrogel can be adjusted by controlling the content of the water-insoluble polymer according to the particular use.

In particular, when the stimuli-responsive hydrogel according to an embodiment is applied to an actuator capable of carrying out expansion/contraction operation as in living skeletal muscles, the content of the water-insoluble polymer is as follows. When the monomer component constituting the stimuli-responsive polymer comprises only a monomer having a stimuli-responsive functional group, the content of the water-insoluble polymer is such that the volume ratio of the water-insoluble polymer to the stimuli-responsive polymer or the monomer having a stimuli-responsive functional group is preferably 100:5 to 100:100 and more preferably 100:10 to 100:60. When the monomer component constituting the stimuli-responsive polymer comprises a monomer having a stimuli-responsive functional group and another copolymerizable monomer having no stimuli-responsive functional group, the content of the water-insoluble polymer is such that the volume ratio of the water-insoluble polymer to the monomer having a stimuli-responsive functional group is preferably 100:5 to 100:100 and more preferably 100:10 to 100:60. When the stimuli-responsive polymer is used as a mixture with another polymer, the content of the water-insoluble polymer is set depending on which case of the above cases the monomer component constituting the stimuli-responsive polymer corresponds to.

The water-insoluble polymer can be contained in any of various states in the stimuli-responsive polymer hydrogel. For example, it may be contained as island-like, separated fine particles or may constitute an “interpenetrating network structure” in which the molecular chain of the hydrogel and the molecular chain of the water-insoluble polymer separate from each other but are entangled with each other.

Next, the method for producing the stimuli-responsive polymer hydrogel according to an embodiment will be described.

Initially, the polymerization of a monomer having a stimuli-responsive functional group with a crosslinker is carried out in a solution of a water-insoluble polymer in an organic solvent to yield an organogel comprising the water-insoluble polymer and a stimuli-responsive polymer.

Next, the organogel is subjected to a treatment selected from drying under reduced pressure, drying by heating, and drying by heating under reduced pressure to remove the organic solvent to thereby yield a dried gel. Next, the dried gel is swelled with water to thereby yield the stimuli-responsive hydrogel according to an embodiment.

By carrying out the crosslinking and polymerization of a monomer having a stimuli-responsive functional group in the presence of a water-insoluble polymer as is described above, the resulting stimuli-responsive polymer and the water-insoluble polymer can be satisfactorily mixed without the need of the procedure of mixing them.

The production method requires an organic solvent that can dissolve or be miscible with both the water-insoluble polymer and the monomer having a stimuli-responsive functional group.

Consequently, the solubility parameters (Sps) of the organic solvent, the water-insoluble polymer, and the monomer having a stimuli-responsive functional group are preferably substantially equivalent, and the difference among the solubility parameters (Sps) is preferably within about ±1.

The organic solvent to be used preferably has a boiling point lower than 150° C., because the organic solvent is removed by any of drying under reduced pressure, drying by heating, and drying by heating under reduced pressure.

If the organic solvent has a high boiling point and cannot be significantly removed by any of drying under reduced pressure, drying by heating, and drying by heating under reduced pressure, the stimuli-responsive hydrogel according to an embodiment can also be prepared in the following manner; the polymerization of a monomer having a stimuli-responsive functional group with a crosslinker is carried out in a solution of a water-insoluble polymer in the organic solvent to yield an organogel comprising the water-insoluble polymer and a stimuli-responsive polymer, and the organogel is immersed in water or a water-containing liquid mixture to thereby carry out solvent exchange.

The volume change of the stimuli-responsive hydrogels according to an embodiment in response to a stimulus can be applied to polymer actuators.

An actuator can be constructed, for example, in the following manner. A pair of electrodes are embedded in a pair of stimuli-responsive polymer hydrogels, respectively, so that an anode is embedded in an acidic polymer hydrogel and a cathode is embedded in a basic polymer hydrogel. The resulting actuator can contrast upon application of a predetermined voltage between the electrodes, and it can expand upon application of a reversed voltage between the electrodes. The actuator serves to drive, for example, the moving elements of robots.

### EXAMPLES

The stimuli-responsive hydrogels according to the present invention will be illustrated with reference to several examples below.

### Example 1

As an organic solvent, N,N-dimethylformamide (DMF) having a solubility parameter (Sp) of 12.1 was prepared.

In 5 ml of DMF were dissolved 1 ml of acrylic acid (AA, having a solubility parameter (Sp) of 12.0) as a monomer having a stimuli-responsive functional group, 0.1 g of N,N′-methylenebisacrylamide (BIS) as a crosslinker, 0.01 g of 2,2′-azobisisobutyronitrile (AIBN) as a polymerization initiator, and 0.476 g of a poly(methyl methacrylate) having a molecular weight of 35×10⁴ (PMMA, having a glass transition temperature (Tg) of 105° C. and a solubility parameter (Sp) of 11.1) to thereby yield an organogel precursor solution.

The 0.476 g of PMMA has a dry volume of 0.4 ml and corresponds to 40% of 1 ml of AA.

Next, the gelation of the above-prepared organogel precursor solution was carried out by placing the precursor solution in a glass tube having an inner diameter of 4 mm and a length of 100 mm, sealing both ends of the glass tube with rubber stoppers, and elevating the temperature to 60° C.

After the gelation, the rubber stoppers were removed from the glass tube, and the organogel in the glass tube was dried by heating at 60° C. under reduced pressure to thereby remove DMF therefrom. The dried gel was immersed in and swelled with ion-exchanged water, and was washed with ion-exchanged water repeatedly. Thus, a pH-responsive poly(acrylic acid) hydrogel containing a water-insoluble polymer PMMA (PAA-PMMA) according to the present invention was obtained.

The above-prepared rod-like pH-responsive poly(acrylic acid) hydrogel containing the water-insoluble polymer (PAA-PMMA) was measured in breaking strength with a tensile tester and was found to have a tensile breaking strength of 0.8 MPa.

The rod-like PAA-PMMA hydrogel was immersed in a 50-mN aqueous sodium hydroxide solution, and the length of the rod (L1) was measured when the swelling reached equilibrium. The rod-like hydrogel was then immersed in a 50-mN aqueous hydrochloric acid solution, and the length of the rod (L2) was measured when the swelling reached equilibrium. The percentage of change in length [(1−L2/L1)×100] was 31%.

### Example 2

As an organic solvent, N,N-dimethylformamide (DMF) having a solubility parameter (Sp) of 12.1 was prepared.

In 5 ml of DMF were dissolved 1 ml of acrylic acid (AA, having a solubility parameter (Sp) of 12.0) as a monomer having a stimuli-responsive functional group, 0.1 g of N,N′-methylenebisacrylamide (BIS) as a crosslinker, 0.01 g of 2,2′-azobisisobutyronitrile (AIBN) as a polymerization initiator, and 0.42 g of a polystyrene having a molecular weight of 23×10⁴ (PS, having a glass transition temperature (Tg) of 78° C. and a solubility parameter (Sp) of 8.6) and thereby yielded an organogel precursor solution.

The 0.42 g of PS has a dry volume of 0.4 ml and corresponds to 40% of 1 ml of AA.

Next, the gelation of the above-prepared organogel precursor solution was carried out by placing the precursor solution in a glass tube having an inner diameter of 4 mm and a length of 100 mm, sealing both ends of the glass tube with rubber stoppers, and elevating the temperature to 60° C.

After the gelation, the rubber stoppers were removed from the glass tube, and the organogel in the glass tube was dried by heating at 60° C. under reduced pressure to thereby remove DMF therefrom. The dried gel was immersed in and swelled with ion-exchanged water, and was washed with ion-exchanged water repeatedly. Thus, a pH-responsive poly(acrylic acid) hydrogel containing the water-insoluble polymer PS (PAA-PS) was obtained.

The above-prepared rod-like pH-responsive poly(acrylic acid) hydrogel containing the water-insoluble polymer PS (PAA-PS) was measured in breaking strength with a tensile tester and was found to have a tensile breaking strength of 0.1 MPa.

The rod-like PAA-PS hydrogel was immersed in a 50-mN aqueous sodium hydroxide solution, and the length of the rod (L1) was measured when the swelling reached equilibrium. The rod-like hydrogel was then immersed in a 50-mN aqueous hydrochloric acid solution, and the length of the rod (L2) was measured when the swelling reached equilibrium. The percentage of change in length [(1−L2/L1)×100] was 25%.

### Example 3

As an organic solvent, N,N-dimethylformamide (DMF) having a solubility parameter (Sp) of 12.1 was prepared.

In 5 ml of DMF were dissolved 1 ml of acrylic acid (AA, having a solubility parameter (Sp) of 12.0) as a monomer having a stimuli-responsive functional group, 0.1 g of N,N′-methylenebisacrylamide (BIS) as a crosslinker, 0.01 g of 2,2′-azobisisobutyronitrile (AIBN) as a polymerization initiator, and 0.712 g of a poly(vinylidene fluoride) having a molecular weight of 27×10⁴ (PVdF, having a glass transition temperature (Tg) of −35° C. and a solubility parameter (Sp) of 11.3) and thereby yielded an organogel precursor solution.

The 0.712 g of PVdF has a dry volume of 0.4 ml and corresponds to 40% of 1 ml of AA.

Next, the gelation of the above-prepared organogel precursor solution was carried out by placing the precursor solution in a glass tube having an inner diameter of 4 mm and a length of 100 mm, sealing both ends of the glass tube with rubber stoppers, and elevating the temperature to 60° C.

After the gelation, the rubber stoppers were removed from the glass tube, and the organogel in the glass tube was dried by heating at 60° C. under reduced pressure to thereby remove DMF therefrom. The dried gel was immersed in and swelled with ion-exchanged water, and was washed with ion-exchanged water repeatedly. Thus, a pH-responsive poly(acrylic acid) hydrogel containing the water-insoluble polymer PVdF (PAA-PVdF) was obtained.

The above-prepared rod-like pH-responsive poly(acrylic acid) hydrogel containing the water-insoluble polymer PVdF (PAA-PVdF) was measured in breaking strength with a tensile tester and was found to have a tensile breaking strength of 2.5 MPa.

The rod-like PAA-PVdF hydrogel was immersed in a 50-mN aqueous sodium hydroxide solution, and the length of the rod (L1) was measured when the swelling reached equilibrium. The rod-like hydrogel was then immersed in a 50-mN aqueous hydrochloric acid solution, and the length of the rod (L2) was measured when the swelling reached equilibrium. The percentage of change in length [(1−L2/L1)×100] was 34%.

### COMPARATIVE EXAMPLE

As an organic solvent, N,N-dimethylformamide (DMF) having a solubility parameter (Sp) of 12.1 was prepared.

In 5 ml of DMF were dissolved 1 ml of acrylic acid (AA, having a solubility parameter (Sp) of 12.0) as a monomer having a stimuli-responsive functional group, 0.1 g of N,N′-methylenebisacrylamide (BIS) as a crosslinker, and 0.01 g of 2,2′-azobisisobutyronitrile (AIBN) as a polymerization initiator and thereby yielded an organogel precursor solution. Namely, a water-insoluble polymer was not incorporated in this comparative example.

Next, the gelation of the above-prepared organogel precursor solution was carried out by placing the precursor solution in a glass tube having an inner diameter of 4 mm and a length of 100 mm, sealing both ends of the glass tube with rubber stoppers, and elevating the temperature to 60° C.

After the gelation, the rubber stoppers were removed from the glass tube, and the organogel in the glass tube was dried by heating at 60° C. under reduced pressure to thereby remove DMF therefrom. The dried gel was immersed in and swelled with ion-exchanged water, and was washed with ion-exchanged water repeatedly. Thus, a pH-responsive poly(acrylic acid) hydrogel (PAA) was obtained.

The above-prepared rod-like pH-responsive poly(acrylic acid) hydrogel (PAA) was measured in breaking strength with a tensile tester and was found to have a tensile breaking strength of 0.01 MPa.

The rod-like PAA hydrogel was immersed in a 50-mN aqueous sodium hydroxide solution, and the length of the rod (L1) was measured when the swelling reached equilibrium. The rod-like hydrogel was then immersed in a 50-mN aqueous hydrochloric acid solution, and the length of the rod (L2) was measured when the swelling reached equilibrium. The percentage of change in length [(1−L2/L1)×100] was 45%.

The tensile breaking strength is improved by incorporating a water-insoluble polymer into a pH-responsive hydrogel, as is obvious from the results of

### Examples 1 to 3 and Comparative Example

The samples according to EXAMPLES 1 to 3 use water-insoluble polymers as reinforcers and thereby ensure practically sufficient stimuli-responsive function (percentage of change in length), because the reinforcers do not become hydrated in the hydrogels and can exhibit high reinforcing effects even in a small amount.

The sample according to EXAMPLE 3 has a set glass transition temperature Tg of the water-insoluble polymer as the reinforcer lower than the working temperature of the stimuli-responsive polymer hydrogel. In this sample, the water-insoluble polymer can be in a rubbery state upon use, thereby has higher motility of the molecular chain, achieves stress dispersion more effectively, and yields higher reinforcing effect than the samples according to EXAMPLES 1 and 2 in which the water-insoluble polymer is in a glassy state upon use.

The samples according to EXAMPLES 1 and 3 selectively use materials for the organic solvent, the water-insoluble polymer, and the monomer having a stimuli-responsive functional group so that the difference in their solubility parameter (Sp) is within ±1. These samples maintain the stimuli-responsive polymer and the water-insoluble polymer in a more satisfactorily mixed state and combine high reinforcing effect and practically sufficient stimuli-responsive function more satisfactorily than the sample according to EXAMPLE 2.

The sample according to EXAMPLE 3 selectively uses, as the water-insoluble polymer, a material having a glass transition temperature Tg lower than the working temperature of the stimuli-responsive hydrogel. In addition, it selectively uses materials for the organic solvent, the water-insoluble polymer, and the monomer having a stimuli-responsive functional group so that the difference in their solubility parameter (Sp) is within ±1. This sample therefore permits the water-insoluble polymer to be in a rubbery state under working conditions of the stimuli-responsive polymer hydrogel, thereby has a higher motility of the molecular chain of water-insoluble polymer, and enables sufficient stress dispersion. In addition, it permits the stimuli-responsive polymer and the water-insoluble polymer in a satisfactorily mixed state. Thus, the sample can combine very high reinforcing effect and practically sufficient stimuli-responsive function.

1-7. (canceled)

8. A stimuli-responsive polymer hydrogel comprising:
a water-insoluble polymer as a phase separation structure;
wherein the polymer hydrogel is capable of gelating as a result of absorbing and swelling with water and capable of changing its degree of swelling and/or volume in response to a stimulus.

9. The stimuli-responsive polymer hydrogel according to claim 1, wherein the water-insoluble polymer is a polymer without a cross-linking point.

10. The stimuli-responsive polymer hydrogel according to claim 1, wherein the water-insoluble polymer has a glass transition temperature lower than a working temperature of the stimuli-responsive polymer hydrogel, and wherein the water-insoluble polymer has a rubbery characteristic at the working temperature.

11. The stimuli-responsive polymer hydrogel according to claim 1, wherein the stimulus is a change in pH, and wherein the stimuli-responsive polymer hydrogel changes volume in response to the pH change.

12. A method for producing a stimuli-responsive polymer hydrogel, comprising:
polymerizing a monomer having a stimuli-responsive functional group with a crosslinker in a solution of a water-insoluble polymer in an organic solvent to yield an organogel comprising the water-insoluble polymer and a stimuli-responsive polymer;
subjecting the organogel to a treatment to remove the organic solvent to thereby yield a dried gel, the treatment selected from the group consisting of: drying under reduced pressure; drying by heating; and drying by heating under reduced pressure; and
allowing the dried gel to swell with water to thereby yield a hydrogel.

13. A method for producing a stimuli-responsive hydrogel, comprising:
carrying out the polymerization of a monomer having a stimuli-responsive functional group with a crosslinker in a solution of a water-insoluble polymer in an organic solvent to yield an organogel comprising the water-insoluble polymer and a stimuli-responsive polymer; and
immersing the organogel in water or a water-containing liquid mixture to thereby yield a hydrogel.

14. A polymer actuator comprising:
a stimuli-responsive polymer hydrogel capable of gelating as a result of absorbing and swelling with water and capable of changing its volume in response to a stimulus,
wherein the stimuli-responsive polymer hydrogel includes a water-insoluble polymer as a phase separation structure.
